(19) United States
(12) Patent Application Publication
(10) Pub. No.: US 2017/0002511 A1
(43) Pub. Date: Jan. 5, 2017

(54) HYDROGELLING FIBERS AND FIBER STRUCTURES

(71) Applicant: Carl Freudenberg KG, Weinheim (DE)

(72) Inventors: Samuel Duncker-Rakow, Darmstadt (DE); Bernd Schlesselmann, Weinheim (DE); Katharina Krampfl, Oehringen (DE)

(21) Appl. No.: 15/038,740

(22) PCT Filed: Sep. 18, 2014
(86) PCT No.: PCT/EP2014/002525

§ 371 Date: May 24, 2016

(30) Foreign Application Priority Data
Nov. 28, 2013 (DE) 10 2013 019 888.7

(51) Int. Cl. D06M 13/192; D01F 6/14; D01D 10/02; A61L 15/42; A61L 15/24

(52) U.S. Cl. CPC D06M 13/192; A61L 15/42; A61L 15/24; D01D 10/02; D01F 6/14; D06M 2101/24

(57) ABSTRACT

A method for producing hydrogelling fibers or fiber structures, involving tempering fibers or fiber structures composed of a first fiber raw material comprising water-soluble polyvinyl alcohol and/or water-soluble polyvinyl alcohol copolymer for a predetermined tempering duration at a predetermined tempering temperature that is greater than a glass transition temperature and/or less than a melting temperature of the first fiber raw material used, such that the fibers are cross-linked, wherein the fibers or fiber structures are provided with an acid catalyst before the tempering.

### CROSS-REFERENCE TO RELATED APPLICATIONS

This application is a U.S. national stage application under 35 U.S.C. §371 of International Application No. PCT/EP2014/002525, filed on Sep. 18, 2014, and claims benefit to German Patent Application No. DE 10 2013 019 888.7, filed on Nov. 28, 2013. The International application was published in German on Jun. 4, 2015, as WO 2015/078538 A1 under PCT Article 21(2).

### FIELD

The present invention relates to hydrogelling fibers or one-, two- or three-dimensional fibrous structures produced from a first fiber raw material

### BACKGROUND

From WO 01/30407 A1 there is known a method for producing hydrogels for use as wound dressings, with which burns or other skin injuries can be treated. In the method, an aqueous solution of polyvinyl alcohol, agar-agar and at least one further natural polymer is prepared. This solution is filled into disposable plastics containers at 70-80° C., and the containers are sealed. After cooling to room temperature, the samples filled into the disposable plastics containers are irradiated and thus sterilized.

WO 2005/103097 A1 describes hydrogels which comprise at least one polyvinyl alcohol star-shaped polymer. The hydrogels are produced by repeatedly freezing and thawing an aqueous solution comprising at least one polyvinyl alcohol star-shaped polymer and optionally further components. Such hydrogels can further be produced by the action of ionising radiation on an aqueous solution comprising at least one polyvinyl alcohol star-shaped polymer or by reacting a polyvinyl alcohol star-shaped polymer in aqueous solution with cross-linking reagents.

Disadvantages of the methods currently known for producing hydrogels, in particular for treating wounds, are the complex production method and the problematic further processing of the hydrogels, as well as the possible occurrence of chemical impurities in the hydrogels cross-linked, for example, by a chemical reaction. In addition, in contrast to fibers and fibrous structures, hydrogel films have a smaller surface area, so that they have a lower absorption capacity for water or aqueous solutions. In particular when using polyvinyl alcohol as a raw material for hydrogels, it must be ensured that the polyvinyl alcohol has a high degree of cross-linking, since otherwise solutions of the polyvinyl alcohol in the liquid medium form instead of hydrogels. High stability of the polyvinyl alcohol to water or aqueous solutions is consequently desirable. Moreover, polyvinyl alcohol and polyvinyl alcohol copolymers are distinguished by high biocompatibility and biotolerability, so that there is an increasing need for further forms of hydrogels or hydrogelling materials with polyvinyl alcohol and/or polyvinyl alcohol copolymers which are additionally inexpensive and simple to produce and can be processed further without problems.

J Mater Sci (2010) 45:2456-2465 describes a method for producing nanofibers and fibrous structures of polyvinyl alcohols by means of electrospinning, in which the fibers or fibrous structures are stabilized with respect to aqueous solutions by means of heat treatment. Fibrous structures of nanofibers have the disadvantage that, owing to their fiber diameter of from 244 to 270 nm, they have very low strength and elongation at maximum force as well as only a low absorption capacity. In addition, the described fibers are stabilized with respect to aqueous solutions, so that they do not have gelling properties, do not swell in aqueous solution and are not suitable for trapping water in the fiber (lack of retention).

Wound dressings of hydrogelling fibers, for example of carboxymethylcellulose or modified cellulose, are known in principle. However, they form with the exudate a very soft hydrogel with low maximum force and elongation at maximum force. This has the disadvantage that they are difficult to remove in one piece from the wound or wound cavity. It is thus possible for residues of the wound dressing to remain in the wound, which residues must be removed again by laborious cleaning of the wound. This means an increased outlay in terms of time and thus also cost for the hospital staff. In addition, the wound can be harmed or damaged again by the cleaning.

Fibers of polyvinyl alcohol are available commercially in various types and comprise polyvinyl alcohol of different water solubility. Water-insoluble types of polyvinyl alcohols are, for example, the high strength polyvinyl alcohol fibers having a particularly high maximum force in the dry state. Commercial water-soluble fibers of polyvinyl alcohol are obtainable with a temperature-dependent water solubility, for example water solubility above a temperature of 90° C., of 70° C., of 60° C., of 40° C. or 20° C. Although commercial fibers of polyvinyl alcohol can vary in terms of their water solubility, they do not have hydrogelling properties and thus also do not retain water.

From WO 2012/048768 there are known fibers and fibrous structures, produced from water-soluble polyvinyl alcohol, which have been cross-linked by tempering fibers or fibrous structures of a first fiber raw material comprising water-soluble polyvinyl alcohol and/or water-soluble polyvinyl alcohol copolymer at a predetermined tempering temperature. These fibers and fibrous structures can be produced comparatively simply and inexpensively and can be processed further without problems. They are used, for example, as bandages or wound dressings. They are distinguished by increased stability, in particular a high maximum force and elongation at maximum force in the hydrogelled state, so that they can be removed from the wound or wound cavity in one piece. However, practical tests have shown that comparatively long tempering times, for example of more than 4 hours, are required in this method in order to provide the fibers or fibrous structures with sufficient stability.

### SUMMARY

An aspect of the invention provides a method for producing fibers or fibrous structures, configured to be hydrogelling, the method comprising: tempering one or more fibers or fibrous structures of a first fiber raw material, the first fiber raw material comprising water-soluble polyvinyl alcohol and/or water-soluble polyvinyl alcohol copolymer, for a predetermined tempering time at a predetermined tempering temperature, the predetermined tempering temperature being higher than a glass transition temperature and/or lower than a melting temperature of the first fiber raw material that is used, so that the one or more fibers are cross-linked, wherein the one or more fibers or fibrous structures comprise an acid catalyst, provided prior to tempering.

### BRIEF DESCRIPTION OF THE DRAWINGS

The present invention will be described in even greater detail below based on the exemplary FIGURE. The invention is not limited to the exemplary embodiments. All features described and/or illustrated herein can be used alone or combined in different combinations in embodiments of the invention. The features and advantages of various embodiments of the present invention will become apparent by reading the following detailed description with reference to the attached drawings which illustrate the following:

FIG. 1 shows a punch used for punching out the test samples.

### DETAILED DESCRIPTION

An aspect of the present invention relates to hydrogelling fibers or one-, two- or three-dimensional fibrous structures produced from a first fiber raw material, wherein the first fiber raw material comprises water-soluble polyvinyl alcohol and/or polyvinyl alcohol copolymer, and to an associated production method. An aspect of the invention relates further to the use of such fibers or fibrous structures for wound care, in particular in products for medical care, such as wound dressings, as well as in hygiene and cosmetic products or the like. An aspect of the invention relates further to products for medical care, in particular wound dressings, as well as to hygiene and cosmetic products.

The fibers or fibrous structures according to an aspect the invention can advantageously be used in direct contact with the wound or with the body. Wound care products produced from the fibers or fibrous structures according to the invention swell in contact with aqueous solutions or wound exudate and form a stable hydrogel which has an extraordinarily high maximum force and elongation at maximum force. As a result, wound dressings comprising the fibers or fibrous structures according to the invention can be removed from the wound in one piece. In addition, the fibers or fibrous structures according to the invention have a particularly high absorption capacity and a particularly high retention for aqueous solutions.

An aspect of present invention is concerned with the object of developing further the method known from WO 2012/048768 so that the tempering times can be reduced. It is further to be possible to further process and/or use the fibers or fibrous structures obtained by this method without problems. In addition, bandages or wound dressings produced from the fibers or fibrous structures according to the invention are to have high stability, in particular high maximum force and elongation at maximum force in the hydrogelled state, so that they can be removed from the wound or wound cavity in one piece.

According to the invention, the object is achieved by the subject-matter of the independent claims. Advantageous embodiments are the subject of the dependent claims.

Surprisingly, it has been found that the required tempering time and/or tempering temperature in a method of the type mentioned at the outset can be reduced significantly if the fibers or fibrous structures are provided with an acid catalyst prior to tempering. It has thus been found that, with this procedure, tempering times of less than 0.5 hour are sufficient to provide the fibers or fibrous structures with sufficient stability. Practical tests have shown that particularly good results can be achieved with tempering times of from 1 minute to 0.5 hour, preferably from 1 minute to 15 minutes, yet more preferably from 1 minute to 10 minutes, yet more preferably from 1 minute to 5 minutes, and in particular from 1 minute to 3 minutes.

The preferred tempering temperatures are in the range of from 100 to 210° C., preferably from 130 to 190° C. and in particular from 150 to 180° C. This effect is presumably attributable to the fact that the cross-linking reaction which takes place upon tempering is at least in part a chemical reaction which can be accelerated by acid catalysis.

A very wide variety of acids can be used as the acid catalyst. A Lewis acid and/or a protonic acid is preferably used as the acid catalyst. The protonic acid can have one or more acid functions and can preferably be an organic acid, yet more preferably a C₁₋₁₀-carboxylic acid, in particular a C₂₋₆-carboxylic acid. The carboxylic acid can be branched or unbranched. According to one embodiment of the invention, the carboxylic acid is unsubstituted. According to an alternative embodiment of the invention, the carboxylic acid has one or more substituents. Preferred substituents are alcohol, amino and/or halogen radicals.

The advantage of the use of lower carboxylic acids, for example C₂₋₆-carboxylic acids, is that they are volatile and/or form volatile thermal decomposition products and can thus be removed from the substrate during the tempering without leaving a residue. The following acids have been found to be particularly suitable for this purpose: acetic acid, formic acid, propionic acid and citric acid.

In some cases, however, it can also be expedient to use non-volatile acids in order thus to provide the substrate with a desired property. The following acids have been found to be particularly suitable for this purpose: benzoic acid, para-toluenesulfonic acid. By using these acids, the product can be provided with acidic properties. For example, pH regulation in cosmetic applications can thereby be achieved. Acids selected from the group consisting of di- or tri-valent metal ions, in particular Zn(II) and Al(III), have been found to be particularly suitable Lewis acids

In the method according to the invention, the fibers or fibrous structures of the first fiber raw material can be provided with the acid catalyst in various ways. It has been found to be expedient in particular to apply the acid catalyst from a solution or suspension. The solution or suspension comprises the acid catalyst as well as a solvent or solvent mixture which is expediently so chosen that the fibers or fibrous structures are insoluble or have only low solubility therein. Preference is given to the use of a solvent in which the fibers or fibrous structures have a solubility at 20° C. of less than 1 g per liter, preferably from 0 to 0.6 g per liter, in particular from 0 to 0.3 g per liter. There have been found to be suitable, for example, solvents based on alcohol, preferably selected from the group consisting of ethanol, methanol, isopropyl alcohol.

The solution or suspension can be applied to the fibers or fibrous structures, for example, by foularding, spraying, slop-padding and/or foam impregnation. Application by means of a foulard has been found to be particularly suitable. This type of application has the advantage that treated fibrous structures can be soaked completely.

According to one embodiment of the invention, the solvent is removed by drying, for example in an oven arranged downstream of the application apparatus, after the solution or suspension containing the acid catalyst has been applied. This has the advantage that solvent extraction does not have to be provided in downstream method steps. Alternatively, removal of the solvent can also take place at the same time as tempering of the fibers or fibrous structure. This has the advantage that the number of method steps can be reduced.

The amount of acid catalyst applied to the fibers or fibrous structure is advantageously from 0.01 to 15 wt. %, preferably from 0.05 to 10 wt. %, in particular from 0.1 to 1 wt. %, in each case based on the weight of the fibers.

According to a further preferred embodiment of the invention, a bonding process to produce a one-, two- or three-dimensional fibrous structure, in particular to produce a nonwoven, is carried out before the acid catalyst is applied. This procedure is advantageous since bonded fibers can be provided significantly more simply with the acid catalyst. Unbonded fibers, on the other hand, can be provided with the acid catalyst only with difficulty, since they adhere to one another, form clumps and therefore can be coated evenly only with difficulty.

By means of the method according to the invention, fibers or fibrous structures comprising water-soluble polyvinyl alcohol can be treated by tempering so that they form a stable hydrogel with aqueous solutions or wound exudate, in particular with a 0.9 percent strength aqueous sodium chloride solution (physiological saline) or with an aqueous solution according to test solution A specified in DIN 13726-1 in point 3.2.2.3, which hydrogel has a very high maximum force and elongation at maximum force. In addition, such fibers or fibrous structures have high stability to water or aqueous solutions. The fibers or fibrous structures according to the invention are further distinguished by a high absorption capacity and a high retention for water or aqueous solutions, in particular 0.9 percent strength aqueous sodium chloride solution (physiological saline) or an aqueous solution according to test solution A specified in DIN 13726-1 in point 3.2.2.3.

In a further aspect of the invention there are proposed fibers or fibrous structures, one-, two- or three-dimensional, which can be produced by the method described above. These fibers or fibrous structures can be produced from fibers of a first fiber raw material, wherein the first fiber raw material comprises water-soluble polyvinyl alcohol and/or polyvinyl alcohol copolymer and wherein the fiber raw material is cross-linked and configured to be hydrogelling by tempering for a predetermined tempering time at a predetermined tempering temperature which is higher than the glass transition temperature and/or lower than the melting or decomposition temperature of the first fiber raw material that is used. By means of this treatment, the fiber raw material is stabilized, and the fibers or fibrous structures produced from the fiber raw material are in particular stabilized with respect to aqueous solutions so that they exhibit significantly reduced solubility in aqueous solution. At the same time, the fibers or fibrous structures form a stable hydrogel with aqueous solutions.

Within the meaning of this invention, tempering is understood as being a process in which the fiber raw material, preferably in the form of fibers or fibrous structures, is heated for a predetermined time at a predetermined temperature, preferably at atmospheric pressure and in a gas atmosphere, in particular an air atmosphere. The fiber raw material is expediently tempered in the form of fibers or a fibrous structure in the dry state, advantageously with a residual moisture content of less than 10 wt. %, yet more preferably of less than 5 wt. %, yet more preferably of less than 3 wt. %. The fibers or fibrous structures are expediently first brought to the predetermined temperature and then maintained at that predetermined temperature for the predetermined time. Temperature fluctuations of at least +/−10%, in particular +/−5% and preferably +/−1%, which occur thereby can be tolerated. In addition, as much air as desired can supplied or removed during the tempering process, and the air can be circulated in the tempering region by various means (for example circulating air, through-air). Other process gases such as nitrogen or oxygen can additionally be fed in during the tempering process in order to influence the tempering process, and thus the properties of the fibers or fibrous structures, in a desired manner.

Particularly preferably, the tempering process in the case of two-dimensional fibrous structures or nonwovens is carried out with through-air in a belt dryer. By means of the through-air, the tempering time can be reduced considerably as compared with the tempering time with pure circulating air.

The fibers or fibrous structures can advantageously be so cross-linked by means of tempering that they have greater solubility stability towards water. Moreover, as a result of the tempering, the fibers or fibrous structures acquire the ability to form a stable hydrogel with water or aqueous solutions, in particular with 0.9 percent strength sodium chloride solution or with a solution according to test solution A specified in DIN 13726-1 in point 3.2.2.3, which hydrogel is distinguished by a particularly high maximum force and elongation at maximum force.

In addition, impurities or residues, such as, for example, spinning aids, brighteners, solvents or the like, can be significantly reduced by the tempering or even reduced to a concentration below the respective detection limit. Furthermore, the fibers or fibrous structures according to the invention have a high absorption capacity and a high retention for water, aqueous solutions, in particular for a 0.9 wt. % aqueous sodium chloride solution or for a solution according to test solution A specified in DIN 13726-1 in point 3.2.2.3, and/or for wound exudate.

The fibers or fibrous structures can thus have a retention of over 70%, preferably from 70% to 100%, for water and/or aqueous solutions. In the case of fibers and/or one-dimensional as well as two-dimensional fibrous structures, the relative retention for 0.9 percent strength sodium chloride solution or for a solution according to test solution A specified in DIN 13726-1 in point 3.2.2.3 is over 70%, yet more preferably over 80%, yet more preferably over 85%, yet more preferably from 85% to 100%.

The fibers or fibrous structures can additionally have a relative absorption capacity for 0.9 percent strength sodium chloride solution or for a solution according to test solution A specified in DIN 13726-1 in point 3.2.2.3 of from 4 to 30 g/g. In the case of fibers and/or one-dimensional as well as two-dimensional fibrous structures, the relative absorption capacity for 0.9 percent strength sodium chloride solution or for a solution according to test solution A specified in DIN 13726-1 in point 3.2.2.3 is from 4 to 30 g/g, particularly preferably from 4 to 25 g/g, yet more preferably from 5 to 20 g/g, yet more preferably from 7 to 20 g/g. Accordingly, there can advantageously be produced toxicologically harmless and biocompatible fibers or fibrous structures, as well as gels, in particular hydrogels, which can be produced therefrom.

Fibers are understood as being a structure which is thin and flexible in relation to its length. Fibers have a small diameter and can be assembled with one another by corresponding bonding processes to form fibrous structures. A fibrous structure can thus comprise a plurality of fibers. A distinction can be made between one-, two- and three-dimensional fibrous structures. A one-dimensional fibrous structure has a small width and a small height in comparison to its length. A two-dimensional fibrous structure has a small height in comparison to its length and width. Three-dimensional fibrous structures are to be understood as being fibrous structures which comprise a plurality of layers of two-dimensional fibrous structures. The individual layers of the three-dimensional fibrous structure can be connected together by bonding processes described hereinbelow or by other means.

Filaments can be produced from polymers by means of the dry or wet spinning process, and spunlaid nonwovens can be produced by means of the spunlaid process. Filaments can thereby be regarded as one-dimensional fibrous structures, while spunlaid nonwovens can constitute two-dimensional fibrous structures. Staple fibers, which can be classified as one-dimensional fibrous structures, can be produced by cutting and/or crimping the filaments. Staple fiber yarns can be produced from staple fibers by twisting yarn. They can be understood as being one-dimensional fibrous structures. Yarns composed of filaments can be formed from one filament (monofilament yarn) or from a plurality of filaments (multifilament yarn). They can likewise be regarded as one-dimensional fibrous structures. Mixed yarns can be produced by spinning more than one different staple fiber or natural fiber. Yarns such as natural fiber yarns, staple fiber yarns or filament yarns or mixed yarns can be processed further by means of textile engineering processes such as weaving, weft knitting, warp knitting, stitching, laying or stitching to form, for example, woven fabrics, warp-knitted fabrics, non-crimped fabrics or weft-knitted fabrics. Woven fabrics, warp-knitted fabrics, non-crimped fabrics or weft-knitted fabrics can be regarded as two-dimensional fibrous structures. Staple fiber nonwovens or airlaid nonwovens, which can likewise be regarded as two-dimensional fibrous structures, can be produced from staple fibers by means of nonwoven processes such as carding or the airlaid process. Preference is given according to the invention to the use of water-soluble staple fibers which are laid to form a staple fiber nonwoven by means of carding.

Unbonded nonwovens, for example staple fiber or spun nonwovens, can be bonded to form nonwovens by bonding processes. Calendering, for example, can be used as the bonding process. In that process, the unbonded nonwovens are guided between rollers, sealing surfaces arranged on the rollers producing in the nonwovens seals which penetrate the nonwovens at least partially. If point-like seals are produced, the bonding process is referred to as a PS (point seal) bonding process. However, the formation of linear seals or seals over the entire surface is also possible. A further bonding process which can be used is hot air bonding in a through-air dryer, bonds being produced in this process by fusion at the points of contact of the fibers. Furthermore, the use of binders or binding agents is likewise conceivable, the fibers in this case being bonded together via bridges of binders or binding agents. Mechanical bonding processes in particular can also be used, such as, for example, the needle bonding process, in which bonding is carried out by means of needles. Furthermore, fulling or felting or the like is also conceivable. It is also possible to use a combination of a plurality of bonding processes. The needle bonding process and/or the PS bonding process are preferably used.

By means of the tempering, the water-soluble fibers of polyvinyl alcohol or the fibrous structures comprising the water-soluble fibers of polyvinyl alcohol can be cross-linked. Accordingly, both the fibers themselves and the fibrous structures can be so changed by tempering that they have a higher stability to water, in particular to a 0.9 percent strength aqueous sodium chloride solution or to a solution according to test solution A specified in DIN 13726-1 in point 3.2.2.3.

The tempered fibers or the fibrous structures produced therefrom preferably have a soluble content of from 1% to 30%, preferably from 1% to 25%, yet more preferably from 1% to 20% and yet more preferably from 1% to 15%, in 0.9% strength aqueous sodium chloride solution or in a solution according to test solution A specified in DIN 13726-1 in point 3.2.2.3.

Moreover, tempering advantageously imparts to the fibers or the fibrous structure the property of forming with water or with the solutions mentioned above a stable hydrogel having a high maximum force and elongation at maximum force. “Hydrogelling” is to be understood as meaning the ability to form a hydrogel which contains as the liquid phase water or an aqueous solution, particularly preferably a 0.9 percent strength aqueous sodium chloride solution or a solution according to test solution A specified in DIN 13726-1 in point 3.2.2.3.

A hydrogel is a hydrophilic polymeric network swollen in water. In particular, a hydrogel is to be understood as being a system of at least a solid phase and a liquid phase, wherein the solid phase forms a three-dimensional network whose pores can be filled by aqueous solution and thereby swell. The two phases can penetrate one another completely and consequently a gel, in comparison to a sponge, is able to store a liquid phase more stably towards pressure, for example. Moreover, a hydrogel has a high retention for aqueous solutions.

Fibers or fibrous structures according to the invention are configured to be hydrogelling and consequently have an outstanding binding ability and retention for aqueous phases. They are preferably applied in the dry state to the wound, or wound cavities are filled therewith. They form stable hydrogels with the wound exudate and thus create an optimal wound climate for wound healing without sticking to the wound. Such moist wound treatment can assist with the healing process. Owing to the high maximum force and elongation at maximum force of the hydrogel formed with the wound exudate, the fibers or fibrous structures can be removed from the wound or wound cavity in one piece.

Likewise for moist wound treatment, the fibers or fibrous structures according to the invention can be used in hydrogelled form when provided with a liquid phase. There is used as the aqueous phase preferably water and particularly preferably a 0.9 percent strength aqueous sodium chloride solution, Ringer's solution or solutions comprising active ingredients or a solution according to test solution A specified in DIN 13726-1 in point 3.2.2.3.

Polyvinyl alcohols are polymers and can be produced from polyvinyl acetate by hydrolysis. The technical properties of the polyvinyl alcohol, such as in particular its water solubility, depend inter alia on the production method, on the molar mass and on the remaining content of acetyl groups (degree of hydrolysis). As the molar mass and degree of hydrolysis fall, the solubility in water increases. Depending on the molar mass and degree of hydrolysis, the polyvinyl alcohols have different water solubility. Thus, some types of polyvinyl alcohol dissolve in water only at an elevated temperature (for example above 90° C.). Fibers of polyvinyl alcohol are conventionally stretched to a multiple of their original length during their production and can thereby also be heated (stretching temperature) in order to increase the crystallinity and strength of the fibers. The formation of intermolecular hydrogen bonds is made possible by parallel orientation of the molecule chains. The water solubility of the polyvinyl alcohol fibers can also be adjusted.

According to the invention, the untempered fibers of polyvinyl alcohol used as the first fiber raw material can be water-soluble in an excess of water even below a temperature of 50° C., preferably below 40° C., particularly preferably below 30° C., yet more preferably below 25° C., it naturally being possible for the untempered fibers also to be water-soluble above those values. The untempered fibers can further also be water-soluble above 15° C. and/or above 20° C. In particular, the untempered fibers can be water-soluble in a range of between 0° C. and 150° C. or between 5° C. and 100° C. or between 10° C. and 100° C. or between 15° C. and 100° C. or between 20° C. and 100° C., water-soluble being understood as meaning that the fibers dissolve in an excess of water to the extent of at least 70%, preferably to the extent of more than 80%, yet more preferably to the extent of more than 90% and in particular to the extent of more than 95%, and in particular to the extent of 100%.

The polyvinyl alcohol used for the production of the fibers of polyvinyl alcohol can be modified by copolymerization with other monomers (for example polyethylene vinyl alcohol) or by the incorporation of functional groups, whereby further physical and also chemical properties are optionally purposively incorporated into the fibers. Thus, in the case of the use of, for example, polyethylene vinyl alcohol, the number of OH groups is reduced.

There can be used as polyvinyl alcohol copolymers preferably polyethylene vinyl alcohol, polyvinyl alcohol styrene, polyvinyl alcohol vinyl acetate, polyvinyl alcohol vinylpyrrolidone, polyvinyl alcohol ethylene glycol and/or polyvinyl alcohol, particularly preferably polyethylene vinyl alcohol, polyvinyl alcohol vinyl acetate, polyvinyl alcohol vinylpyrrolidone, polyvinyl alcohol vinylamine, polyvinyl alcohol acrylate, polyvinyl alcohol acrylamide, polyvinyl alcohol ethylene glycol. The polyvinyl alcohol copolymers can be in the form of block copolymers and/or graft copolymers and/or block and graft copolymers, random or alternating systems and any mixtures with one another. The content of other monomer units in the polyvinyl alcohol is not more than 30 wt. %, preferably from 1 to 30%, yet more preferably from 5 to 15%, in each case based on the total number of monomer units in the polyvinyl alcohol copolymer.

However, other functional groups can also be introduced into the polyvinyl alcohol and/or into the fibers or into the fibrous structure, for example by substitution or polymer-analogous reactions. There come into consideration as functional groups in particular carboxylic acids, unsaturated carboxylic acids, such as methacrylic acids, acrylic acids, peroxycarboxylic acids, sulfonic acids, carboxylic acid esters, sulfonic acid esters, aldehydes, thioaldehydes, ketones, thioketones, amines, ethers, thioethers, isocyanates, thiocyanates, nitro groups. The content of other functional groups in the polyvinyl alcohol is not more than 30%, preferably from 1 to 30%, yet more preferably from 5 to 15%, in each case based on the number of OH groups in the polyvinyl alcohol.

Furthermore, the first fiber raw material can be in the form of a physical mixture between the water-soluble polyvinyl alcohol and at least one other polymer (polymer blend). The content of water-soluble polyvinyl alcohol in the polymer blend is at least 70 wt. %, based on the total mass of the polymer blend.

Advantageously, the resulting polymer blend has different physical properties and optionally also chemical properties as compared with the polymers used. The properties of the polymer blend are usually a sum of the properties of the polymers used. Accordingly, a choice of first fiber raw materials can be expanded further by the use of polymer blends. In order to form such a polymer blend there can be used and added to the water-soluble polyvinyl alcohol gelling further polymers, such as, for example, alginates, cellulose ethers, such as carboxymethylcelluloses, methyl-, ethyl-celluloses, hydroxymethylcelluloses, hydroxyethylcelluloses, hydroxyalkylmethylcelluloses, hydroxypropylcelluloses, cellulose esters, such as cellulose acetate, oxidized celluloses, bacterial celluloses, cellulose carbonates, gelatins, collagens, starches, hyaluronic acids, pectins, agar, polyacrylates, polyvinylamines, polyvinyl acetates, polyethylene glycols, polyethylene oxides, polyvinylpyrrolidones, polyurethanes, or non-gelling further polymers, such as, for example, polyolefins, cellulose, cellulose derivatives, regenerated cellulose such as viscose, polyamides, polyacrylonitriles, polyvinyl chlorides, chitosans, polylactides, polyglycolides, polyester amides, polycaprolactones, polyhexamethylene terephthalates, polyhydroxy butyrates, polyhydroxy valerates or polyesters. The above-mentioned blends can be used in the form of homopolymers or copolymers. Block copolymers and/or graft copolymers and/or block and graft copolymers, random or alternating systems and any mixtures with one another can also be used.

Alginates are understood as being the salts of alginic acid, a natural polymer, occurring in algae, of the two uronic acids α-L-glucuronic acid and β-D-mannuronic acid, which are linked 1,4-glycosidically. The term alginate includes E401, E402, E403, E404 and E405 (PGA). The term polyolefins includes PE, PB, PIB and PP. The term polyamides includes PA6, PA6.6, PA6/6.6, PA6.10, PA6.12, PA69, PA612, PA11, PA12, PA46, PA1212 and PA6/12. The term cellulose also includes regenerated cellulose such as viscose, as well as cellulose derivatives and chemically and/or physically modified cellulose. The term polyester includes PBT, BC, PET, PEN and UP.

The polyvinyl alcohol which is used for producing the fibers of polyvinyl alcohol or of which the polyvinyl alcohol fibers are made can be used with various degrees of hydrolysis and mean molar masses.

The degree of hydrolysis of the polyvinyl alcohol is in particular more than 70%, preferably above 75%, yet more preferably above 80% and up to 100%.

The weight-average molar mass of the polyvinyl alcohol is in particular in the range of from 20,000 to 200,000 g/mol, preferably in the range of from 30,000 to 170,000 g/mol, particularly preferably in the range of from 40,000 to 150,000 g/mol, yet more preferably in the range of from 50,000 to 140,000 g/mol, yet more preferably in the range of from 70,000 to 120,000 g/mol.

The number-average molar mass of the polyvinyl alcohol is in particular in the range of from 10,000 to 120,000 g/mol, preferably in the range of from 20,000 to 100,000 g/mol, particularly preferably in the range of from 20,000 to 80,000 g/mol, yet more preferably in the range of from 25,000 to 70,000 g/mol.

Fibers of a first fiber raw material having a fiber titer of from 0.5 to 12 dtex can be used. They are used preferably with a fiber titer of from 1 to 8 dtex, particularly preferably with a fiber titer of from 1.4 to 7 dtex and yet more preferably with a fiber titer of from 1.4 to 4 dtex. dtex or decitex is to be understood as meaning the weight in grams of the fibers at an optional theoretical length of 10,000 m. Fibers with an individual titer of less than 0.5 dtex are less suitable.

The fibers of a first fiber raw material can have a length of from 30 to 100 mm. They are used preferably with a length of from 30 to 90 mm, particularly preferably with a length of from 30 to 80 mm and yet more preferably with a length of from 35 to 70 mm.

The fibers of the first fiber raw material are in particular so-called staple fibers, which are used for the production of staple fiber nonwovens.

The fibers or fibrous structures can additionally comprise further fibers of at least a second fiber raw material. The second fiber raw material can be non-gelling or gelling. Non-gelling or gelling fibers can accordingly be used as further fibers. By the use of further fibers, a desired behavior of the fibers or fibrous structures can advantageously purposively be improved. Thus, by using the further fibers, the absorption capacity of the fibrous structures can be increased further and the shrinkage of the fibrous structure in aqueous solution can be reduced.

There can be used as the further fiber raw material for the further fibers polyesters, such as polyethylene terephthalate, water-insoluble polyvinyl alcohol, water-soluble polyvinyl alcohol which is water-soluble above a temperature of 50° C., polyolefins, such as polyethylene or polypropylene, cellulose, cellulose derivatives, regenerated cellulose, such as viscose, polyamides, polyacrylonitriles, chitosans, elastanes, polyvinyl chlorides, polylactides, polyglycolides, polyester amides, polycaprolactones, natural plant fibers, alginates, modified chitosan, cellulose ethers, such as carboxymethylcelluloses, methyl-, ethyl-celluloses, hydroxymethylcelluloses, hydroxyethylcelluloses, hydroxyalkylmethylcelluloses, hydroxypropylcelluloses, cellulose esters, such as cellulose acetate, oxidized celluloses, bacterial celluloses, cellulose carbonates, gelatins, collagens, starches, hyaluronic acids, pectins, agar, polyvinylamines, polyvinyl acetates, polyethylene glycols, polyethylene oxides, polyvinylpyrrolidones, polyurethanes and/or polyacrylates. The second fiber raw materials listed can be used both in the form of homopolymers and in the form of copolymers. Block copolymers and/or graft copolymers and/or block and graft copolymers, random or alternating systems and any mixtures with one another can also be used.

The simultaneous use of gelling and non-gelling further fibers or of mixtures of different further fibers is also possible. Preference is given to the use of further fibers of polyamide, polyester, water-insoluble polyvinyl alcohol or polyvinyl alcohol which dissolves above a temperature of 50° C., polyacrylate, polyacrylic acid, and yet more preferably of polyester or water-insoluble polyvinyl alcohol or polyvinyl alcohol which dissolves above a temperature of 50° C. and/or mixtures thereof.

The further fibers can also be produced from a second fiber raw material in the form of a polymer blend. The advantages already indicated above for the first fiber raw material are obtained for the further fibers.

The fibers of the first fiber raw material or of the further fiber raw material can also be used in the form of a bicomponent fiber and/or multicomponent fiber. The bicomponent fibers and/or multicomponent fibers can be in geometric forms such as core-shell, side-by-side, pie- or orange-type, matrix with fibrils.

The bicomponent fibers and/or multicomponent fibers of the further fiber raw material can be used for thermal bonding of the nonwovens. When these fibers are heated, thermal bonding of the nonwoven takes place. In a core-shell fiber, for example, the shell component melts and thus bonds the nonwoven. There can be used as bicomponent fibers and/or multicomponent fibers of the further fiber raw material of polyethylene/polypropylene, polyethylene/polyester, co-polyester/polyethylene terephthalate, polyamide 6/polyamide 6.6, polybutylene terephthalate/polyethylene terephthalate.

By the use of further fibers, the absorption capacity for water, in particular for a 0.9 percent strength aqueous sodium chloride solution or a solution according to test solution A specified in DIN 13726-1 in point 3.2.2.3, can advantageously be increased significantly as compared with fibrous structures without further fibers, since a gel-blocking effect, which prevents the further absorption of water from a predetermined saturation, in particular of a 0.9 percent strength sodium chloride solution or of a solution according to test solution A specified in DIN 13726-1 in point 3.2.2.3, can be reduced in particular by means of the non-gelling fibers. In addition, the shrinkage in aqueous solution of the fibrous structures comprising fibers of the first fiber raw material can be reduced significantly by adding further fibers.

The shrinkage of at least two-dimensional fibrous structures can be determined by punching out pieces having a size of 10.0 cm×10.0 cm (surface area 1) and immersing them in a 0.9% aqueous sodium chloride solution or a solution according to test solution A specified in DIN 13726-1 in point 3.2.2.3. The pieces which have been punched out and immersed are removed from the solution and allowed to drip for 2 minutes. The size of the pieces is then measured (surface area 2). The shrinkage of the nonwovens can then be calculated according to the following formula:

$$Shrinkage [ \% ] = 100 - \frac{Surface area 2 [ {cm}^{2} ]}{Surface area 1 [ {cm}^{2} ]} * 100$$

The content of further fibers in the fibrous structures can be from 1 to 70 wt. %. The content is preferably from 1 to 65 wt. %, particularly preferably from 5 to 60 wt. %, yet more preferably from 10 to 50 wt. %, yet more preferably between 15 and 40 wt. %.

The further fibers can have a fiber titer of from 0.5 to 12 dtex. They are preferably used with a fiber titer of from 1 to 8 dtex, particularly preferably with a fiber titer of from 1.4 to 7 dtex and yet more preferably with a fiber titer of from 1.4 to 4 dtex. dtex or decitex is to be understood as meaning the weight in grams of the fibers at an optionally theoretical length of 10,000 m. Fibers with an individual titer of less than 0.5 dtex are less suitable.

The further fibers can have a length of from 30 to 100 mm. They are used preferably with a length of from 30 to 90 mm, particularly preferably with a length of from 30 to 80 mm and yet more preferably with a length of from 35 to 70 mm.

The further fibers of the further fiber raw material are in particular staple fibers, which are used to produce staple fiber nonwovens.

Furthermore, the fibers or fibrous structures can additionally comprise additives. There can be used as additives pharmacological active ingredients or medicaments, such as antibiotics, analgesics, anti-infectives, anti-inflammatory agents, agents promoting wound healing or the like, antimicrobial, antibacterial or antiviral agents, haemostatic agents, enzymes, amino acids, antioxidants, peptides and/or peptide sequences, polysaccharides (for example chitosan), growth factors (for example purines, pyrimidines), living cells, tricalcium phosphate, hydroxyapatite, in particular hydroxyapatite nanoparticles, odour-absorbing additives such as activated charcoal, cyclodextrins, metals such as silver, gold, copper, zinc, carbon compounds, such as activated charcoal, graphite or the like, cosmetic active ingredients, vitamins and/or processing aids such as surface-active substances, wetting agents, brighteners, antistatics.

By the use of at least one additive, the fibers or fibrous structures can additionally advantageously be provided with further physical, chemical and biological properties. For example, providing the fibers or fibrous structures with silver or silver salts or antimicrobial agents such as polyhexanide (polyhexamethylene biguanide), chlorhexidine, cetylpyridinium chloride, benzalkonium chloride, Medihoney, PVP-iodine, hydrogen peroxide, 8-quinolinol, chloramine, ethacridine lactate, nitrofural or octenidine (N-octyl-1-[10-(4-octyliminopyridin-1-yl)decyl]pyridin-4-imine), permits an antibacterial action of the fibers or fibrous structures.

For example, the fibers or fibrous structures can be provided with an ethanolic solution which comprises an antimicrobial agent. The fibers or fibrous structures are preferably provided with an ethanolic solution which comprises an antimicrobial agent, such as polyhexanide, octenidine or silver salts, by means of a foulard. However, any other coating methods also come into consideration. In addition, the fibers or fibrous structures can be provided with an aqueous solution which comprises the antimicrobial agent. Preferably, in the case of application from aqueous solution, a controlled amount of water is used, in the presence of which the fibers or fibrous structures do not irreversibly hydrogel and change in terms of their morphological structure. In particular, coating methods such as foam application, kiss coating or the like come into consideration.

The fibrous structures according to the invention can have a weight per unit area, measured according to DIN EN 29073, of from 10 to 1000 g/m². In the case of two-dimensional fibrous structures, the weight per unit area is preferably from 10 to 700 g/m², particularly preferably from 20 to 600 g/m², yet more preferably from 50 to 500 g/m², yet more preferably from 70 to 450 g/m², yet more preferably from 80 to 400 g/m², yet more preferably from 90 to 350 g/m², yet more preferably from 100 to 300 g/m², yet more preferably from 120 to 240 g/m².

In the case of two- or three-dimensional fibrous structures, the thickness of the fibrous structure is preferably in the range of from 0.2 to 10 mm, preferably in the range of from 0.5 to 8 mm, yet more preferably in the range of from 0.7 to 7 mm, yet more preferably in the range of from 0.8 to 6 mm, yet more preferably in the range of from 0.9 to 5 mm, particularly preferably in the range of from 1.0 to 4 mm.

Two- or three-dimensional fibrous structures are preferably bonded thermally or mechanically. They are particularly preferably bonded mechanically by needling. The punch density is preferably in the range of from 70 to 200 punches per square centimeter, particularly preferably in the range of from 70 to 170 punches per square centimeter, particularly preferably in the range of from 80 to 150 punches per square centimeter, particularly preferably in the range of from 100 to 150 punches per square centimeter.

The fibrous structures according to the invention can have a particularly high maximum force in the hydrogelled state, both in the longitudinal direction and in the transverse direction of the fibrous structure. For example, fibrous structures according to the invention which have a weight per unit area of from 140 to 220 g/m² and have been mechanically bonded by needling, for example with a punch density of from 100 to 150 punches per square centimeter, have a maximum force in the hydrogelled state of above 0.3 N/2 cm. The preferred maximum force in the hydrogelled state is above 0.4 N/2 cm, yet more preferably above 0.5 N/2 cm, yet more preferably above 0.8 N/2 cm, yet more preferably above 1.0 N/2 cm, yet more preferably above 1.5 N/2 cm, yet more preferably above 2.0 N/2 cm and/or below 50 N/2 cm, and/or below 40 N/2 cm, and/or below 35 N/2 cm. Accordingly, a maximum force in the hydrogelled state is preferably in the range of from 0.3 N/2 cm to 50 N/2 cm, yet more preferably from 0.4 N/2 cm to 40 N/2 cm, yet more preferably from 0.5 N/2 cm to 30 N/2 cm, yet more preferably from 0.8 N/2 cm to 25 N/2 cm, yet more preferably from 1 N/2 cm to 25 N/2 cm, yet more preferably from 1.5 N/2 cm to 25 N/2 cm, yet more preferably from 2 N/2 cm to 25 N/2 cm.

The fibrous structures according to the invention can have a particularly high elongation at maximum force in the hydrogelled state, both in the longitudinal direction and in the transverse direction of the fibrous structure. The preferred elongation at maximum force in the hydrogelled state is from 20 to 300%, particularly preferably from 30 to 250%, yet more preferably from 50 to 200%, yet more preferably from 70 to 200%, yet more preferably from 80 to 200%, yet more preferably from 90 to 190%, yet more preferably from 90 to 180%. For example, fibrous structures according to the invention which have a weight per unit area of from 140 to 220 g/m² and have been mechanically bonded by needling, for example with a punch density of from 100 to 150 punches per square centimeter, have the above-mentioned elongation at maximum force values.

As described above, the fibers or fibrous structures which are configured to be hydrogelling can be produced by tempering fibers or fibrous structures of a first water-soluble fiber raw material comprising polyvinyl alcohol and/or unsubstituted or partially unsubstituted polyvinyl alcohol copolymer which have been provided with an acid catalyst for a predetermined tempering time at a predetermined tempering temperature which is preferably higher than a glass transition temperature and/or lower than a melting temperature of the first fiber raw material that is used, so that the fibers are cross-linked.

It is advantageously possible by means of this very simple process to produce fibers or fiber structures which have hydrogelling properties quickly and efficiently. Only a small number of process steps are necessary to stabilize the fibers or fibrous structures. In addition, any impurities, such as, for example, brighteners, spinning aids or solvents, which may be contained in the fibers or fibrous structures can be removed by the tempering.

The predetermined tempering temperature is preferably so chosen that it is higher than the glass transition temperature of the first fiber raw material that is used. In addition, the predetermined tempering temperature can be so chosen that it is lower than the melting temperature of the first fiber raw material that is used. If a plurality of fibers of different fiber raw materials are used, the predetermined temperature is preferably so chosen that it is below the melting temperature or decomposition temperature of preferably all the fiber raw materials that are used.

In many cases, tempering temperatures in a temperature range of from 85 to 220° C., particularly preferably from 100 to 200° C., yet more preferably from 120° C. to 190° C., yet more preferably between 130° C. and 180° C., most particularly preferably between 140° C. and 180° C., yet more preferably between 150° C. and 175° C., have been found to be expedient.

Practical tests have shown that particularly good results can be achieved with tempering times of from 1 minute to 0.5 hour, preferably from 1 minute to 15 minutes, yet more preferably from 1 minute to 10 minutes, yet more preferably from 1 minute to 5 minutes, and in particular from 1 minute to 3 minutes.

By choosing such tempering temperatures and tempering times, the cross-linking according to the invention of the fibers or fibrous structures can be carried out in a manner which is particularly gentle for the fibers or fibrous structures. In addition, by choosing those tempering conditions, the properties of the fibers or fibrous structures can optimally be adjusted. Thus, as a result of choosing those tempering conditions, the fibers or fibrous structures have a high absorption capacity and retention as well as a very high maximum force and elongation at maximum force in the hydrogelled state. By varying the tempering temperatures and tempering times, the cross-linking can be controlled to be configured differently, so that the cross-linked fibers or fibrous structures optionally have different properties. By choosing those tempering conditions, any impurities, such as solvent residues or fiber adjuvants and fiber processing aids, such as brighteners, wetting agents, antistatics, which may be present can be removed from the fibers or fibrous structures, even to a content that is no longer detectable. This is advantageous in particular for the use of the fibers or fibrous structures in wound dressings, since the above-mentioned impurities or fiber adjuvants/processing aids can be toxicologically harmful.

A method for obtaining one-, two- or three-dimensional fibrous structures can be carried out in particular before the tempering. The fibrous structure in question can thereby be produced from the fibers, for example by means of an above-described method.

The fibers or fibrous structures can advantageously be brought into a desired form by such a bonding process and can be bonded in that form.

In addition, further fibers of at least a second fiber raw material can also be added.

Furthermore, an after-treatment can be carried out. The addition of processing aids is additionally possible, in particular before the bonding process. The addition of, for example, above-described additives can likewise be carried out.

As a possible after-treatment there can be carried out post-bonding, sterilization, such as, for example, radiosterilization or sterilization with ethylene oxide, irradiation, coating, finishing, the application of brighteners, chemical modification, or further processing, such as, for example, raschel knitting, introduction of reinforcing fibers.

A particularly preferred after-treatment of the fibers or fibrous structures is plasma treatment in order in particular to increase the hydrophilicity of the fibers or fibrous structures. Plasma is a mixture of neutral and charged particles. In special cases, only charged particles are present. Different species, such as electrons, cations, anions, neutral atoms, neutral or charged molecules, are present in the plasma. By means of the active particles contained in the plasma, surfaces such as, for example, fibers or nonwovens can be modified. Different effects can be achieved thereby, such as, for example, a change in the surface by plasma etching, plasma activation or plasma polymerization. In the case of plasma activation, the surface is activated by means of a plasma with the addition of oxygen. In plasma polymerization, further organic precursor compounds are introduced into the process chamber.

The fibers or fiber adjuvants can be rendered hydrophobic by the tempering, since the fiber adjuvants and fiber processing aids can be reduced by the tempering. The plasma treatment can be carried out both under atmospheric pressure and under a vacuum, in particular with the addition of oxygen. Further substances such as acrylic acid can also be added during the plasma treatment.

A preferred after-treatment is additionally the sterilization of the fibers or fibrous structures for use in particular for wound dressings. The sterilization is preferably carried out by radiosterilization or by sterilization with ethylene oxide. Properties such as, for example, absorption capacity and/or maximum force and elongation at maximum force in the hydrogelled state can be influenced positively by the sterilization.

The individual method steps of tempering, bonding, addition of further fibers, addition of additives, addition of processing aids and after-treatment can be repeated several times in any order. It has been found to be expedient to temper the fibers or fibrous structures at least once for a predetermined tempering time at a predetermined tempering temperature.

There can be used as processing aids brighteners, antistatic agents, surfactants, stabilizers, lubricants or the like.

In a preferred variant of the production method, the fibers of a first fiber raw material, in particular water-soluble polyvinyl alcohol staple fibers, are tempered for the purpose of cross-linking in particular for from 10 minutes to 7 hours at a predetermined tempering temperature which is above the glass transition temperature and below the melting temperature of the fibers of a first fiber raw material. Further fibers, in particular non-gelling fibers, particularly preferably polyester fibers, can subsequently optionally be added in an amount by weight of from 10 to 50 wt. %. A two-dimensional fibrous structure, such as, for example, a nonwoven, can then be produced by means of a bonding process from the fibers so produced, optionally using processing aids, such as, for example, brighteners or antistatic agents.

In another preferred variant of the production method, fibers of a first fiber raw material can optionally be mixed with further fibers of a second fiber raw material, wherein the amount of further fibers is preferably from 10 to 50 wt. %. It is, however, also possible to use only fibers of a first fiber raw material. Polyvinyl alcohol fibers are preferably used as fibers of a first fiber raw material and polyester fibers are preferably used as further fibers of a second fiber raw material. A two-dimensional fibrous structure such as, for example, a nonwoven can be produced from those fibers by means of a bonding process. The two-dimensional fibrous structure so produced can subsequently be tempered at a tempering temperature above the glass transition temperature and below the melting temperature of the fibers of a first fiber raw material. A two-dimensional fibrous structure so produced can optionally be subjected to after-treatment.

In a further aspect of the invention there is proposed the use of fibers or fibrous structures as described hereinbefore, wherein such fibers or fibrous structures are used in particular in the production of materials for medical applications, in particular for wound dressings and bandages, and in particular for the production of wound dressings for the field of modern wound care. The fibers or fibrous structures can additionally be used in the production of other materials for medical applications, such as suture materials, implants, tissue engineering scaffolds, transdermal patches, drug delivery products, carrier materials or ostomy products. Also possible is the use thereof in the production of carrier materials, insulating materials, filter materials for the production of hygiene, cosmetic, household products, technical absorber products, such as cable sheathing, products for the foodstuffs sector, such as food packaging. Hygiene products can be understood as being inter alia feminine hygiene products, nappies and incontinence products. Household products also include cleaning materials.

The advantages mentioned hereinbefore inter alia are obtained for the particular use.

As a further aspect of the invention there is proposed a bandage or a wound dressing comprising fibers or fibrous structures as described hereinbefore. Such fibers or fibrous structures can preferably be used in the field of modern wound care, in particular for modern (moist) wound treatment. In modern wound care, the wound dressings establish an optimal moist wound climate, owing to which the wound is able to heal more quickly. Modern wound care is used to treat wounds which are difficult to heal, such as chronic wounds, which can be caused, for example, by pressure or bedsores (decubitus), diabetes, circulatory disorders, metabolic diseases, vascular diseases such as venous insufficiency, or low immunity.

The fibers or fibrous structures according to the invention on the one hand have a high absorption capacity for aqueous solutions and are thus able to absorb and trap the wound exudate. On the other hand, by absorbing the wound exudate, the fibers or fibrous structures form a hydrogel, which traps the fluid firmly and retains it even under pressure, which arises, for example, through applying a bandage. In addition, the formation of the hydrogel creates a moist wound climate, which promotes wound healing. The hydrogelled fibers or fibrous structures adapt to the structure of the wound surface and can be used in particular also for the treatment of wound cavities. As a result of their high maximum force and elongation at maximum force, the hydrogelled fibers or fibrous structures can easily be removed from the wound or wound cavity in one piece, without damaging it.

Such bandages or wound dressings can also be used analogously to conventional bandages or wound dressings, such as, for example, gauze bandages, but have the advantageous hydrogelling properties, so that advantageously improved wound care can be achieved by means of the bandages or wound dressings according to the invention.

### Implementation of the Invention

### Methods and Measuring Methods

It will be shown hereinbelow how different parameters which can be used to characterize the fibers or fibrous structures according to the invention are to be determined in accordance with the invention:

1) Determination of the Thickness of the Two-Dimensional Fibrous Structures and/or Nonwoven

In accordance with DIN EN ISO 9073-2, but without conditioning

2) Determination of the Weight Per Unit Area of the Two-Dimensional Fibrous Structures and/or Nonwoven

In accordance with DIN EN 29073, but without conditioning

### 3) Determination of the Absorption Capacity of Fibers

A 600 ml glass beaker is filled with 300 ml of 0.9% strength sodium chloride solution (0.9 g of sodium chloride dissolved in 100 ml of distilled water) or with a solution according to test solution A specified in DIN 13726-1 in point 3.2.2.3. 0.40 g (dry fiber weight: m_{dry}) of the fibers is stirred into the solution. The fibers are left in the glass beaker for 10 minutes, with occasional stirring by means of a glass rod. The time is recorded by means of a stopwatch. A pre-tared metal screen (32 mesh) is placed onto a 2000 ml glass beaker. The entire contents of the 600 ml glass beaker are poured over the metal screen. The fibers are allowed to drip from the metal screen for 5 minutes. The weight of the metal screen including the fibers is determined. The tare of the metal screen is subtracted from the weight. The fiber weight of the hydrogelled fibers is obtained (m_{wet}).

The absorption capacity of the fibers is determined by means of the following formula:

$$Relative absorption capacity [ ℊ \text{/} ℊ ] = \frac{{m}_{wet} - {m}_{dry}}{{m}_{dry}}$$

where
m_{wet} is the mass of the test sample and the absorbed liquid at the end of the test in g
m_{dry} is the mass of the dry test sample in g.

### 4) Determination of the Absorption Capacity of Two-Dimensional Fibrous Structures or Nonowovens on the Basis of DIN EN ISO 9073-6

The absorption capacity is tested on the basis of DIN EN ISO 9073-6; absorption of liquids.

A 0.9% strength sodium chloride solution (0.9 g of sodium chloride in 100 ml of distilled water) or test solution A according to DIN 13726-1 point 3.2.2.3 is used as the specified liquid (test medium) according to point 5.2.7 of DIN EN ISO 9073-6.

The test medium used is specified with the respective measuring result.

The test samples (size 10*10 cm) are prepared and the determination is carried out analogously to DIN EN ISO 9073-6, but without conditioning.

In addition, in a departure from the standard, the absorption capacity was determined after two different absorption times:
1) absorption capacity after 1 minute: in accordance with the standard, the test samples are immersed in the test medium for 1 minute and allowed to drip for 2 minutes
2) absorption capacity after 1 hour: the test samples are immersed in the test medium for 1 hour and allowed to drip for 2 minutes.

The absorption of liquid (LAC) in percent is calculated according to DIN EN ISO 9073-6 by means of the following formula:

$$LAC [ \% ] = \frac{{m}_{n} - {m}_{k}}{{m}_{k}} \times 100$$

where
mₖ is the mass of the dry test sample in g
mₙ is the mass of the test sample and the absorbed liquid at the end of the test in g.

The relative absorption in g/g is calculated as follows:

$$Relative absorption [ ℊ \text{/} ℊ ] = \frac{{m}_{n} - {m}_{k}}{{m}_{k}}$$

The absolute absorption in g/m² is calculated as follows:

Absolute absorption [g/m²]=relative absorption [g/g]×weight per unit area [g/m²]

After determination of the absorption capacity after 1 hour, the hydrogelled test samples are used further for determining the retention of two-dimensional fibrous structures and/or nonwovens (point 5) and for determining the soluble content of two-dimensional fibrous structures and/or nonwovens (point 6).

### 5) Determination of the Retention of Two-Dimensional Fibrous Structures or Nonwovens

The determination is carried out using the hydrogelled test samples after determination of the absorption capacity (point 4) after 1 hour (absorption capacity after 1 hour); in addition, the calculated values of the masses of the dry test samples, which were calculated during the determination of the absorption capacity, are used: mₖ is the mass of the dry test sample in g.

The test samples are in each case placed on a flat metal mesh having a size of 15×15 cm, which is placed over a bowl so that liquid from the test sample is able to run into the bowl.

The test sample is subjected to a weight, which exerts a pressure of 40 mmHg flat on the entire surface of the test sample (this corresponds to a weight of 5.434 kg on an area of 100 cm²) for a period of 2 minutes. The weight of the test sample is then weighed accurately (mₚᵣₑₛₛᵤᵣₑ).

The relative retention in g/g is calculated as follows:

$$Relative retention [ \frac{ℊ}{ℊ} ] = \frac{{m}_{pressure} - {m}_{k}}{{m}_{k}}$$

The retention in percent is calculated as follows:

$$Retention [ \% ] = \frac{Relative retention}{Relative absorption after 1 hour} * 100$$

### 6) Determination of the Soluble Content of Two-Dimensional Fibrous Structures or Nonwovens

The determination is carried out using the hydrogelled test samples after determination of the absorption capacity (point 4) after 1 hour (absorption capacity after 1 hour); in addition, the calculated values of the masses of the dry test samples, which were calculated during the determination of the absorption capacity, are used: mₖ is the mass of the dry test sample in g.

The hydrogelled test sample is placed in a tared 100 ml glass beaker (m_{glass beaker}). The glass beaker containing the test sample is placed in a commercial laboratory drying cabinet with circulating air at a temperature of 70° C., and the hydrogelled test sample is thereby dried. After 24 hours, the glass beaker containing the dried test sample is removed from the drying cabinet. After cooling, the weight of the test sample (m_{dry}) is determined, the glass beaker being weighed together with the test sample (mₜₒₜₐₗ) and the weight of the glass beaker being subtracted from the weight:

m_{dry}=mₜₒₜₐₗ−m_{glass beaker}

The soluble content in percent is calculated as follows:

$$Soluble content [ \% ] = 100 - ( \frac{{m}_{dry}}{{m}_{k}} * 100 )$$

### 7) Determination of the Shrinkage of Two-Dimensional Fibrous Structures or Nonwovens

The shrinkage is determined by punching out pieces having a size of 10.0 cm×10.0 cm (surface area 1) and immersing them in a test medium. The test medium is either a 0.9% strength aqueous sodium chloride solution or a test solution A according to DIN 13726-1 point 3.2.2.3. The respective test medium is specified with the measuring result.

The pieces which have been punched out and impregnated are removed from the solution after 1 hour and allowed to drip for 2 minutes. The size of the pieces is then measured (surface area 2). The shrinkage of the nonwovens can then be calculated by means of the following formula:

$$Shrinkage [ \% ] = 100 - ( \frac{Surface area 2 [ {cm}^{2} ]}{Surface area 1 [ {cm}^{2} ]} ) * 100$$

8) Determination of the Maximum Force and Elongation at Maximum Force of Two-Dimensional Fibrous Structures and/or Nonwovens in the Hydrogelled State

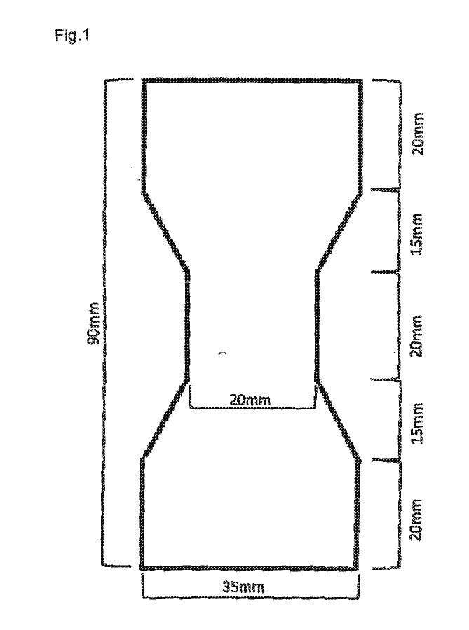

For the determination, pieces of nonwoven of DIN A4 size are punched out and placed in an excess of 0.9% strength sodium chloride solution or test solution A according to DIN 13726-1 point 3.2.2.3. The pieces of nonwoven are removed from the solution after 1 hour. The test samples are punched out of the pieces of hydrogelled nonwoven both in the longitudinal direction (machine direction) of the nonwoven and in the transverse direction of the nonwoven by means of a punch. The punch for punching out the test sample has a length of 90 mm. The width is 35 mm at the top and bottom end. After 20 mm, the punch tapers at both ends to 20 mm (see FIG. 1).

The maximum force and elongation at maximum force are then determined in accordance with EN 29073-03 using a Zwick Z 1.0, but with the following differences:
no conditioning
take-off speed 200 mm/min
different punch (as described above); clamped length adjusted to the length of the punch
different sample preparation: the samples are measured not in the dry state but in the hydrogelled state (preparation of the samples as described above).

The punch used for punching out the test samples is shown in FIG. 1.

### 9) Determination of the Solubility of Water-Soluble Fibers

A 250 ml glass beaker is filled with 200 ml of distilled water and heated by means of a heating plate to the test temperature (temperature at which the fibers of polyvinyl alcohol are water-soluble). Temperature monitoring is by means of a thermometer.

In each case 0.4 g of the fibers is stirred into the 200 ml of tempered water for a short time. The fibers are first left in the glass beaker for 3 minutes without stirring. The contents of the glass beaker are then stirred vigorously for 7 minutes. The time is recorded in each case using a stopwatch. Finally, a visual inspection (with the naked eye) is made to see whether the fibers have dissolved completely. The water solubility is 100 percent when solid fibers or fiber constituents are no longer visible in the solution.

### 10) Determination of Thermodesorption

In the determination of thermodesorption, organic components contained in the fibers are released by heating a sample of fibers or fibrous structures at 150° C. for 20 minutes; the components are focused by means of a cryotrap and then injected into the GC/MS by means of a cooled injection system. A GERSTEL thermodesorption system and a GERSTEL cooled injection system CIS are used. The components that have been released are detected by means of GC/MS. A GC Agilent Technologies 6890N Network GC system, Mass Selective Detector Agilent Technologies 5973 is used thereby.

### 11) Determination of the Wetting Time of Two-Dimensional Fibrous Structures or Nonwovens

The time taken for 1 drop of distilled water to sink into the fibrous structure or nonwoven is measured. The test is carried out with a total of 5 drops and the mean is formed.

### 12) Examination of Fibers or Fibrous Structures by Means of XPS

Measurements by means of XPS (X-ray photoelectron spectroscopy) were carried out using an SSX-100 spectrometer (SSI, US) with monoenergetic Al Kα1,2 excitation (1486.6 eV) in an ultrahigh vacuum (10-9 Torr). The information depth is between 6 and 10 nm. The charge compensation for non-conducting samples is achieved by means of a flood gun. Before the start of the measurement, the samples are stored in a vacuum overnight.

### EXAMPLES

### Comparative Example 1

### Needle-Bonded Nonwovens of Water-Soluble Polyvinyl Alcohol Fibers with Subsequent Thermal Cross-Linking

A needle-bonded nonwoven is produced from water-soluble polyvinyl alcohol staple fibers. The polyvinyl alcohol fibers are water-soluble at a temperature below 25° C. and have a fiber titer of 1.7 or 2.2 dtex, with a staple fiber length of 38 or 51 mm. The polyvinyl alcohol fibers are laid by means of a carding machine to form a nonwoven and are then bonded by needling with a punch density of 100-170 punches per square centimeter. The needle-bonded polyvinyl alcohol nonwovens are tempered at a temperature of 150° C. in order to stabilize the polyvinyl alcohol. The nonwovens are thereby tempered in a commercial laboratory drying cabinet with circulating air. After a tempering time of 2 hours, stability of the polyvinyl alcohol nonwovens is obtained, as is shown by the formation of stable, hydrogelling nonwovens in 0.9% strength aqueous sodium chloride solution or test solution A according to DIN 13726-1 point 3.2.2.3. The stability of the nonwovens increases with the tempering time. At a tempering time of from 2.5 to 7 hours, the nonwovens have high stability. The soluble content of the nonwovens is at a maximum of 20% after 1 hour in test solution A. After the tempering, the relative absorption capacity after 1 minute and 1 hour is determined using test solution A as the test medium. The relative absorption capacity after 1 minute is between 5 and 20 g/g. The relative absorption capacity after 1 hour is between 5 and 20 g/g. The retention of the nonwovens after 1 hour in test solution A was also determined. The retention is between 80 and 100%. In addition, the shrinkage of the bonded nonwovens in test solution A is determined after 1 hour in test solution A. The shrinkage of the polyvinyl alcohol nonwovens is between 30 and 60%, depending on the tempering time and thus on the degree of cross-linking of the nonwovens.

TABLE 1
Example of a needle-bonded nonwoven of water-soluble polyvinyl
alcohol fibers, which has been tempered.

| Parameter | Description/Result |
|---|---|
| Polyvinyl alcohol fibers | 1.5 to 2.2 dtex, |
| | 40-70 mm |
| Temperature at which the polyvinyl alcohol fibers | Below 25° C. |
| are water-soluble | |
| Content of polyvinyl alcohol fibers [%] | 100 |
| Tempering time at 150° C. [min] | 150-300 |
| Type of bonding | Needling |
| Punch density [#/cm²] | 100-170 |
| Weight per unit area [g/m²] | 150-210 |
| Thickness [mm] | 1.5-3.0 |
| Relative absorption capacity [g/g] after 1 minute | 5.0-20.0 |
| in test solution A | |
| Relative absorption capacity [g/g] after 1 hour in | 5.0-20.0 |
| test solution A | |
| Retention [%] after 1 hour in test solution A | 80-100 |
| Soluble content after 1 hour in test solution A [%[ | 0-20 |
| Shrinkage [%] | 30-50 |
| Maximum force in the hydrogelled state [N/2 cm]; | 1-20 |
| longitudinal | |
| Elongation at maximum force in the hydrogelled | 80-300 |
| state [%]; longitudinal | |
| Maximum force in the hydrogelled state [N/2 cm]; | 1-20 |
| transverse | |
| Elongation at maximum force in the hydrogelled | 80-300 |
| state [%]; transverse | |

### Example 2

### Needle-Bonded Nonwovens of Water-Soluble Polyvinyl Alcohol Fibers with Subsequent Acid Treatment and Thermal Cross-Linking

A needle-bonded nonwoven is produced from water-soluble polyvinyl alcohol staple fibers. The polyvinyl alcohol fibers are water-soluble at a temperature below 25° C. and have a fiber titer of 1.7 or 2.2 dtex, with a staple fiber length of 38 or 51 mm. The polyvinyl alcohol fibers are laid by means of a carding machine to form a nonwoven and are then bonded by needling with a punch density of 100-170 punches per square centimeter. The needle-bonded polyvinyl alcohol nonwovens are impregnated with a solution of 1 wt. % citric acid in ethanol in a foulard bath and dried at room temperature in a fume cupboard. The polyvinyl alcohol nonwovens coated with citric acid are tempered at a temperature of 150° C. in order to stabilize the polyvinyl alcohol. The nonwovens are thereby tempered in a commercial laboratory drying cabinet with circulating air. After a tempering time of 10 minutes, stability of the polyvinyl alcohol nonwovens is obtained, as is shown by the formation of stable, hydrogelling nonwovens in 0.9% strength aqueous sodium chloride solution or test solution A according to DIN 13726-1 point 3.2.2.3. The stability of the nonwovens increases with the tempering time. At a tempering time of 30 minutes, the nonwovens have high stability. The soluble content of the nonwovens is at a maximum of 20% after 1 hour in test solution A. After the tempering, the relative absorption capacity after 1 minute and 1 hour is determined using test solution A as the test medium. The relative absorption capacity after 1 minute is between 5 and 20 g/g. The relative absorption capacity after 1 hour is between 5 and 20 g/g. The retention of the nonwovens after 1 hour in test solution A was also determined. The retention is between 80 and 100%. In addition, the shrinkage of the bonded nonwovens in test solution A is determined after 1 hour in test solution A. The shrinkage of the polyvinyl alcohol nonwovens is between 30 and 60%, depending on the tempering time and thus on the degree of cross-linking of the nonwovens.

TABLE 2
Example of a needle-bonded nonwoven of water-soluble polyvinyl
alcohol fibers, which has been tempered.

| Parameter | Description/Result |
|---|---|
| Polyvinyl alcohol fibers | 1.5 to 2.2 dtex, 40-70 mm |
| Temperature at which the polyvinyl | Below 25° C. |
| alcohol fibers are water-soluble | |
| Content of polyvinyl alcohol fibers [%] | 100 |
| Tempering time at 150° C. [min] | 150-300 |
| Type of bonding | Needling |
| Punch density [#/cm²] | 100-170 |
| Weight per unit area [g/m²] | 150-210 |
| Thickness [mm] | 1.5-3.0 |
| Relative absorption capacity [g/g] after | 5.0-20.0 |
| 1 minute in test solution A | |
| Relative absorption capacity [g/g] after | 5.0-20.0 |
| 1 hour in test solution A | |
| Retention [%] after 1 hour in test solution A | 80-100 |
| Soluble content after 1 hour in test | 0-20 |
| solution A [%] | |
| Shrinkage [%] | 30-50 |
| Maximum force in the hydrogelled state | 1-20 |
| [N/2 cm]; longitudinal | |
| Elongation at maximum force in the | 80-300 |
| hydrogelled state [%]; longitudinal | |
| Maximum force in the hydrogelled state | 1-20 |
| [N/2 cm]; transverse | |
| Elongation at maximum force in the | 80-300 |
| hydrogelled state [%]; transverse | |

As is shown by a comparison of Examples 1 and 2, pretreatment of the nonwoven with citric acid as catalyst leads to a significant reduction of the tempering time, the good mechanical and physical properties of the nonwoven in the hydrogelled state being retained.

While the invention has been illustrated and described in detail in the drawings and foregoing description, such illustration and description are to be considered illustrative or exemplary and not restrictive. It will be understood that changes and modifications may be made by those of ordinary skill within the scope of the following claims. In particular, the present invention covers further embodiments with any combination of features from different embodiments described above and below. Additionally, statements made herein characterizing the invention refer to an embodiment of the invention and not necessarily all embodiments.

The terms used in the claims should be construed to have the broadest reasonable interpretation consistent with the foregoing description. For example, the use of the article “a” or “the” in introducing an element should not be interpreted as being exclusive of a plurality of elements. Likewise, the recitation of “or” should be interpreted as being inclusive, such that the recitation of “A or B” is not exclusive of “A and B,” unless it is clear from the context or the foregoing description that only one of A and B is intended. Further, the recitation of “at least one of A, B, and C” should be interpreted as one or more of a group of elements consisting of A, B, and C, and should not be interpreted as requiring at least one of each of the listed elements A, B, and C, regardless of whether A, B, and C are related as categories or otherwise. Moreover, the recitation of “A, B, and/or C” or “at least one of A, B, or C” should be interpreted as including any singular entity from the listed elements, e.g., A, any subset from the listed elements, e.g., A and B, or the entire list of elements A, B, and C.

1: A method for producing fibers or fibrous structures, configured to be hydrogelling, in which the method comprising:
tempering one or more fibers or fibrous structures of a first fiber raw material, the first fiber raw material comprising water-soluble polyvinyl alcohol and/or water-soluble polyvinyl alcohol copolymer, for a predetermined tempering time at a predetermined tempering temperature, the predetermined tempering temperature being higher than a glass transition temperature and/or lower than a melting temperature of the first fiber raw material that is used, so that the one or more fibers are cross-linked,
wherein the one or more fibers or fibrous structures comprise an acid catalyst, provided prior to tempering.

2: The method of claim 1, wherein the acid catalyst is a Lewis acid and/or a protonic acid.

3: The method of claim 2, wherein the protonic acid is present and comprises acetic acid, formic acid, propionic acid, citric acid, benzoic acid, para-toluenesulfonic acid, or a mixture of two or more of any of these,
and/or
wherein the Lewis acid is present and comprises a divalent metal ion, a trivalent metal ion, or a combination of two or more of any of these.

4: The method of claim 1, comprising:
providing the fibers or fibrous structures of the first fiber raw material with the acid catalyst using a solution or suspension of the acid catalyst in a solvent.

5: The method of claim 4, wherein the solution or suspension comprising the acid catalyst is applied to the fibers or fibrous structures by foularding, spraying, slop-padding, and/or foam impregnation.

6: The method of claim 4, wherein the solution or suspension comprises the acid catalyst in an amount of from 0.01 to 10 wt. %, based on a total weight of the solvent.

7: The method of claim 1, wherein an amount of acid catalyst applied to the fibers or fibrous structure is from 0.01 to 15 wt. %, based on a weight of the fibers.

8: The method of claim 4, further comprising:
removing the solvent by drying after the solution or suspension comprising the acid catalyst has been applied.

9: The method of claim 1, wherein the predetermined tempering time is from 1 minute to 0.5 hour.

10: The method of claim 1, further comprising, before the acid catalyst is provided:
carrying out a bonding process to produce a two- or three-dimensional fibrous structure.

11: The method of claim 1, wherein the fibers or fibrous structures further comprise second fibers comprising a second fiber raw material.

12: A fiber or a one-, two- or three-dimensional fibrous structure, configured to be hydrogelling, produced by the method of claim 1, wherein an acid that is not volatile under conditions of the tempering has been used as the acid catalyst.

13: (canceled)

14: Bandages or wound dressings comprising fibers or fibrous structures according to claim 12.

15: The method of claim 1, wherein the first fiber raw material comprises a water-soluble polyvinyl alcohol.

16: The method of claim 1, wherein the first fiber raw material comprises a water-soluble polyvinyl alcohol copolymer.

17: The method of claim 1, wherein the acid catalyst comprises an organic acid.

18: The method of claim 1, wherein the acid catalyst comprises a C₁₋₁₀-carboxylic acid.

19: The method of claim 1, wherein the acid catalyst comprises a C₂₋₆-carboxylic acid.

20: The method of claim 2, wherein the Lewis acid is present and comprises Zn(II) and/or Al(III).
